(19) United States
(12) Patent Application Publication
(10) Pub. No.: US 2008/0293015 A1
(43) Pub. Date: Nov. 27, 2008

(54) Adhesive Liner

(75) Inventors: Eddie Wong, Parsippany, NJ (US); Stuart B. Wilensky, Parsippany, NJ (US); John R. Biddescomb, Parsippany, NJ (US); Stanley J. Lech, Parsippany, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 12/094,652

(22) PCT Filed: Nov. 20, 2006
(86) PCT No.: PCT/US06/61089

§ 371 Date: May 22, 2008

Related U.S. Application Data
(60) Provisional application No. 60/738,904, filed on Nov. 22, 2005.

(51) Int. Cl. A61C 13/12; A61K 6/08; A61Q 11/00

(52) U.S. Cl. 433/180; 523/120; 424/49

(57) ABSTRACT

The present invention provides a denture adhesive liner in the form of an extruded film or sheet, comprising a denture adhesive effective amount of alginate; thermoplastic polymer; and plasticizer, wherein said composition is extrudable into a film that is capable of adhering to a wet mucous surface. The present invention also provides a method for preparing a denture adhesive liner comprising the steps of: preparing a composition of a denture adhesive effective amount of alginate; thermoplastic polymer; and plasticizer; and forming a denture adhesive liner from the composition.

### BACKGROUND OF THE INVENTION

1. Field of the Invention

The present invention relates generally to an adhesive liner for dental prosthesis. More particularly, the present invention relates to an adhesive liner in the form of an extruded film.

2. Description of Related Art

Dentures are substitutes for missing teeth and serve as replacement for all or some of the teeth found in the oral cavity. Over time, even well fitting dentures can become ill fitting due to natural shrinkage and changes in the gum or mucous tissues.

Therefore, adherent creams, liquids, powders, and liners are often used to secure dentures within the mouth. Liners are denture adhesives in the form of a thin film, strip, or wafer with a certain desirable strength and integrity for the liner to be placed in between the prosthesis and the palate or jaw, which swells when in contact with moisture in the mouth without using a support.

Denture adhesive liners disclosed in the prior art are commonly in the form of a woven composite or a multiple layer strip. By way of example, U.S. Pat. No. 3,990,149 discloses an adhesive foil comprising a compressed fiber mat. U.S. Pat. No. 4,880,702 describes a denture stabilizer in the form of a strip consisting of three different layers. U.S. Pat. No. 5,158,825 discloses a denture liner in the form of a non-woven fabric, which is impregnated with an adhesive. U.S. Pat. No. 4,503,116 discloses a denture adhesive liner comprising a laminate of superimposed fiber faced webs, with the fibers of one face of the webs being heat bonded to the fibers on the opposing webs by thermoplastic ethylene oxide polymers. U.S. Pat. No. 5,877,233 discloses a multi-layer denture adhesive liner with at least one non-adhesive self-supporting layer coated by adhesive components. U.S. Patent 2004/0028930 A1 describes how to make an extruded denture adhesive film using a mixed partial salt of a copolymer of maleic acid as an adhesive component.

U.S. Pat. No. 4,373,036 discloses a denture fixative in the form of a single layer strip or film. The single-layer denture adhesive liner disclosed is prepared using a film casting method under vacuum at 55.5° C. from a composition of 43.6 parts of a partially neutralized copolymer of lower alkyl vinyl ether-maleic anhydride, 64.4 parts of hydroxypropyl cellulose, 1 part of color, flavor, antioxidant and preservatives for a total of 120 parts, which is then mixed with 472 parts of water, and 8 parts of glycerin forming a viscous solution. This approach is not economically attractive owing to the amount of time and energy needed to evaporate the water inherently required in the composition to dissolve the hydroxypropyl cellulose into a solution that can be cast onto a moving carrier tape by means of a die, thereafter forming a film that can be dried under vacuum at high temperatures.

There remains a need in the art for a denture liner that has strong adhesive properties yet is efficient to manufacture and easy to use by the consumer. The present invention meets this need.

### SUMMARY OF THE INVENTION

It is an object of the present invention to provide a denture adhesive liner in the form of an extruded film or sheet.

It is another object of the present invention to provide such a denture adhesive liner having an alginate as the sole adhesive component.

It is yet another objective of the present invention to provide such a denture adhesive liner that is a foam film that provides a cushioning effect by adding foaming agents during extrusion.

It is yet another object of the present invention to provide such a denture adhesive liner that is a single layer of film.

It is a further object of the present invention to provide such a denture adhesive liner that is efficiently manufactured.

It is still a further object of the present invention to provide such a denture adhesive liner that has strong adhesion properties.

These and other objects of the present invention are provided by a denture adhesive liner in the form of an extruded film or sheet. The denture adhesive liner may comprise, consist essentially of, or consist of (a) a denture adhesive effective amount of alginate; (b) thermoplastic polymer; and (c) plasticizer, wherein said composition is extrudable into a film that is capable of adhering to a wet mucous surface.

The present invention also provides a method for preparing a denture adhesive liner comprising the steps of: (a) preparing a composition of a denture adhesive effective amount of alginate; thermoplastic polymer; and plasticizer; and (b) forming a denture adhesive liner from the composition.

### DESCRIPTION OF THE DRAWINGS

FIG. 1 is a graph plotting dislodge force in lbs. vs. the hydration time in minutes, comparing the adhesive composition of the present invention with comparative examples;

FIG. 2 is a graph plotting adhesion force and compression force in lbs. vs. time in minutes comparing a 10 mil adhesive liner of the present invention with comparative examples; and

FIG. 3 is a graph plotting adhesion force and compression force in lbs. vs. time in minutes comparing a 13 mil adhesive liner of the present invention with comparative examples.

### DETAILED DESCRIPTION OF THE INVENTION

Surprisingly, it has been discovered that using alginate in an adhesive effective amount as the sole adhesive component in the denture liner according to the present invention results in an adhesive liner with exceptional adhesive properties that is easy and economical to manufacture.

The adhesive component of the present invention is one or more alginates. It has been unexpectedly found that the sole use of one or more alginates as the adhesive component in the adhesive liner of the present invention provides comparable and/or superior adhesion properties to the liner compared to prior art adhesive liners.

Suitable alginates for use in the present invention include, but are not limited to, algin, alginic acid, alginate salts such as for example sodium alginate, potassium alginate, magnesium alginate, calcium alginate, propylene glycol alginate, triethanolamine alginate, or any combinations thereof. Alginates are high molecular weight polysaccharides extracted from kelp. They are salt forms or ester form of linear copolymers of 1,4 linked β-D mannuronic acid and α-L guluronic acid that consist of three distinct polymer segments; Polymanuronic acid segments (M blocks), poly guluronic acid segments (G blocks) and alternating mannuronic acid and guluronic acid units (MG blocks).

The one or more alginates are present in a denture adhesive effective amount. A denture adhesive effective amount means an amount sufficient for a flexible and uniform denture adhesive liner product with good denture adhesive properties, e.g., exhibiting a sufficient cohesive strength to withstand the stresses of mastication which act to rupture the seal and thus dislodge the denture; resistance to degradation under the extreme environmental changes that occur in the oral cavity during such common actions as drinking coffee or other hot beverages; and releasable properties so that the denture wearer may remove the dentures for cleaning and maintenance.

In one embodiment of the present invention, the alginate is present in an amount from about 5 wt. % to about 60 wt. %. In another embodiment, the amount is about 10 wt. % to about 30 wt. %.

The adhesive liner according to the present invention also includes one or more thermoplastic polymers. The one or more thermoplastic polymers are water-soluble. A “thermoplastic polymer” is meant to refer to a material, which is melt processable. As used herein, the term “thermoplastic” refers to a material which softens and/or becomes flexible when exposed to heat and generally returns to its original condition when cooled to room temperature.

As used herein, a material will be considered to be “water-soluble” when it substantially dissolves in excess water to form a solution, thereby losing its initial form and becoming essentially molecularly dispersed throughout the water solution. Typically, the water-soluble material will be free from a substantial degree of crosslinking, as cross-linking tends to render a material water insoluble. Also used herein, the term “water-insoluble” is meant to refer to a material that, when exposed to an excess of water, disperses but does not dissolve. As such, a water-insoluble material generally retains its original identity or physical structure, but in a highly dispersed state and must have sufficient physical integrity to resist flow and fusion with neighboring materials.

Suitable thermoplastic polymer for use in the present invention includes, but is not limited to, polyethylene oxide polymer, hydroxypropylcellulose, hydroxypropylmethylcellulose, or any combinations thereof.

Hydroxypropylcellulose (“HPC”) polymers having a weight average molecular weight between 80,000-1,150,000 are useful for the purposes of this invention. HPC can be used as a thermoplastic polymer component by itself or in combination with other thermoplastic polymer components, i.e., PEO, and the like. HPC is commercially available from Hercules, Inc.(Wilmington, Del.) under the trade name KLUCEL. In one embodiment, HPC is present in the denture adhesive liner composition of the present invention in an amount between 0 and 90 wt. %. In another embodiment, HPC is present between 5 and 20 wt. % in combination with another thermoplastic polymer. In yet a third embodiment, HPC is present as the only thermoplastic polymer in an amount between 60 and 90 wt. %.

Hydroxypropylmethylcellulose (“HPMC”) is another water-soluble cellulose that exhibits thermoplastic polymer processing properties when used in combination with a plasticizer. HPMC can be used as a thermoplastic polymer component by itself or in combination with other thermoplastic polymer components, i.e., PEO, and the like. HPMC is commercially available from Dow Chemical Company of Midland, Mich., USA, under the trade name METHOCEL, which is a HPMC, which is a 2% concentration in water produces a viscosity of 400 cPs.

In one embodiment, HPMC is present in the denture adhesive liner composition of the present invention in an amount between 0 and 90 wt. %. In another embodiment, HPMC is present between 5 and 20 wt. % in combination with another thermoplastic polymer. In yet a third embodiment, HPMC is present as the only thermoplastic polymer in an amount between 60 and 90 wt. %.

If polyethylene oxide polymer (“PEO”) is used, it is desired that the material exhibits a weight average molecular weight that is effective for the denture adhesive liner composition to exhibit sufficient cohesive strength and resistance to degradation properties. In general, if the weight average molecular weight of a PEO polymer is too high, the polymer chains may become heavily entangled which may result in a thermoplastic composition, which is difficult to process. In one embodiment, the PEO polymers suitable for use in the present invention exhibit weight average molecular weights between about 100,000 to about 20,000,000. In another embodiment, the molecular weights are between about 200,000 to about 8,000,000.

In one embodiment, PEO is present in the denture adhesive liner composition of the present invention in an amount between 0 and 90 wt. %. In another embodiment, PEO is present in an amount between 50 and 90 wt. %. In a third embodiment, PEO is present between 30 and 70 wt. % in combination with another thermoplastic polymer.

In yet a fourth embodiment, PEO is present as the only thermoplastic polymer in an amount between 30 and 90 wt. %.

It is generally desired that a toxicologically acceptable compatibilizer or plasticizer be used as an optional third component in an amount sufficient for the denture adhesive composition to exhibit desired extrusion processability properties.

The term “toxicologically acceptable”, as used herein, describes materials which are suitable in their toxicity profile for administration to humans and/or lower animals.

Suitable plasticizers include, but are not limited to, water; polyethylene oxide; polypropylene oxide; glycols such as propylene glycol and polyethylene glycol; polyhydric alcohols such as glycerin and sorbitol; glycerol esters such as glycerol triacetate; fatty acid triglycerides; naphthenic oils; aromatic oils; vegetable oils such as castor oil; or low molecular weight rosin esters, polyterpenes; or any combinations thereof.

If HPMC is used as a thermoplastic polymer by itself, propylene glycol may be used as a plasticizer during melt processing. If PEO is used as a thermoplastic polymer by itself, it has been suggested that water may be used as a fugitive plasticizer for PEO during melt processing.

Plasticizer may be present at a level of from about 1 to about 30 wt. %. Preferably, plasticizer is included in an amount of about 5 to about 25 wt. %, and more preferably about 3 wt. % to about 6 wt. %.

Optionally, the denture adhesive liner composition may comprise one or more therapeutic actives suitable for mucosal or topical administration. The phrase “suitable for mucosal or topical administration”, as used herein, describes agents which are pharmacologically active when absorbed through internal mucosal surfaces of the body such as the oral cavity, or applied to the surfaces of the skin. Therapeutic actives may be present at a level of from about 0 to about 30 wt. % of the total composition.

Therapeutic activities that are useful in these compositions include antimicrobial agents such as iodine, sulfonamides, bisbiguanides, or phenolics; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, cimetidine, eugenol, or hydrocortisone; dentinal desensitizing agents such as potassium nitrate, potassium chloride, strontium chloride or sodium fluoride; anesthetic agents such as lidocaine or benzocaine; anti-fungals; aromatics such as camphor, eucalyptus oil, and aldehyde derivatives such as benzaldehyde; insulin; steroids; and anti-neoplastics. It is recognized that in certain forms of therapy, combinations of these agents in the same delivery system may be useful in order to obtain an optimal effect. Thus, for example, an antimicrobial and an anti-inflammatory agent may be combined in a single delivery system to provide combined effectiveness.

The compositions of the present invention may include one or more antimicrobial agents. Suitable antimicrobial agents for use in the compositions of the present invention may include, but are not limited to, cetylpyridium chloride, domiphen bromide, iodine, sulfonamides, bisbiguanides or phenolics, benzoic acid and sodium benzoate; parabens; sorbic acid and sorbates; propionic acid and propionates; acetic acid and acetates; nitrates and nitrites; sulfur dioxide and sulfites; antibiotics; diethyl pyrocarbonate; epoxides; hydrogen peroxide; phosphates, or any combinations thereof.

The one or more antimicrobial agents may be provided in such an amount of the composition that provides effective antimicrobial properties to the composition. The one or more antimicrobial agents may be present in an amount about 0.0001% to about 2.0% by weight of the composition, preferably 0.001% to about 1.0% by weight, and more preferably from about 0.01% to about 0.5% by weight of the composition.

The compositions of the present invention may include one or more odor adsorbent components. Suitable odor adsorbent components include, but are not limited to, cyclodextrin, activated carbon, sodium bicarbonate, or any combinations thereof. Suitable cyclodextrins for use in the present invention include, but are not limited to, α-cyclodextrins, β-cyclodextrins, γ-cyclodextrins, carboxylmethyl cyclodextrins, hydroxypropyl cyclodextrins, methyl cyclodextrins, sulfated cyclodextrins, quaternary ammonium cyclodextrins, or any combinations thereof.

The one or more odor adsorbent components may be present in an amount about 0 to about 30 wt. %.

The composition may also comprise one or more additional suitable components including, but not limited to, silicon dioxide, antioxidants such as ascorbic acid, colorants, preservatives such as methyl and propyl parabens, thickeners, flavors, fragrances, sensate enhancers, or any combinations thereof.

Suitable components that provide flavor, fragrance, and/or sensate benefit include, but are not limited to, natural or artificial sweetening agents, menthol, menthyl lactate, wintergreen oil, peppermint oil, spearmint oil, leaf alcohol, coolants such as 3-menthoxypropane-1,2-diol and paramenthane, and carboxyamide agents such as N-ethyl-menthane-3-carboxamide.

The one or more additional suitable components may be present in the compositions of the invention in an amount about 0 to about 20 wt. %, based on the total weight of the composition.

The present compositions can be prepared by any of the methods or combination of methods, which follow. The term “mixture”, as used herein, refers to a solution, slurry, or suspension.

In one embodiment, the one or more alginates are mixed together with the film forming component (s) and the optional components in a high shear mixer. Plasticizer is gradually added until a free flowing mixture if formed. Mixing can also be done directly in the extruder such as a twin screw extruder. The mixture is fed into an extruder, e.g., a co-rotating twin screw or single-stage extruder pre-heated to between 70 and 120° C. and then extruded through a die preset at a certain mil gap, e.g., ten mil gap. The extruded film product may be pressed smooth in a hydraulic press or flat-roller or other suitable means, then die-cut to desired shape and size with a stamping machine.

Suitably, the denture liner of the present invention may be extruded as a multilayer film using a multi-layer co-extrusion machine. However, while possible, this is not necessary considering the additional costs. The resulting product has one or more layers being the denture liner composition of the present invention, and one or more layers prepared from a non-adhesive material such as plastic, microcrystalline wax, cloth, fleece, and the like.

A denture adhesive foam liner can be made by injecting foaming agent including, but not limited to, carbon dioxide gas during extrusion.

The following examples further describe and demonstrate embodiments within the scope of the present invention. The examples are given solely for the purpose of illustration and are not to be construed as limitations of the present invention.

### EXAMPLES

TABLE 1
Examples of Liners According to Present Invention

| | Example A | Example B |
|---|---|---|
| Component | (wt. %) | (wt. %) |
| Polyethylene oxide (Polyox) | 0 | 0 |
| Hydroxypropylcellulose (HPC) | 75.8 | 76 |
| MVE/MA Na/Mg/Zn Salt (Triple Salt 251) | 0 | 0 |
| MVE/MA Na/Ca Salt (Double Salt 418) | 0 | 0 |
| Carboxymethylcellulose Sodium | 0 | 0 |
| Sodium Alginate | 17.5 | 17.5 |
| Silicon Dioxide | 2.5 | 2.5 |
| Ascorbic Acid | 0.2 | 0 |
| Polyethylene Glycol 400 | 4 | 4 |

TABLE 2
Comparative Liner Examples

| | Ex. C | Ex. D | Ex. E | Ex. F |
|---|---|---|---|---|
| Component | (wt. %) | (wt. %) | (wt. %) | (wt. %) |
| Polyethylene oxide (Polyox) | 0 | 0 | 5 | 0 |
| Hydroxypropylcellulose (HPC) | 78.3 | 78.3 | 70.8 | 93.3 |
| MVE/MA Na/Mg/Zn Salt | 0 | 15 | 0 | 0 |
| (Triple Salt 251) | | | | |
| MVE/MA Na/Ca Salt | 15 | 0 | 15 | 0 |
| (Double Salt 418) | | | | |
| Carboxymethylcellulose Sodium | 0 | 0 | 2.5 | 0 |
| Sodium Alginate | 0 | 0 | 0 | 0 |
| Silicon Dioxide | 2.5 | 2.5 | 2.5 | 2.5 |
| Ascorbic Acid | 0.2 | 0.2 | 0.2 | 0.2 |
| Polyethylene Glycol 400 | 4 | 4 | 4 | 4 |

To prepare the above-referenced liners, a base powder blend is created by blending all of the ingredients except for the plasticizer together. The base powder blend is introduced to an extrusion process where the plasticizer is injected to the powder blend during extrusion. The powder blend was formulated using a ribbon blender.

A conical twin screw extruder with three heating zones, an injection port for the plasticizer and a 4″ lip die was used. Process temperatures ranged from about 85° C. at the powder feed station to about 110° C. at the die lip station. The extruder speed was about 2 kg per hour.

The resulting film is flexible, uniform and has a translucent white to off-white coloration. The film has a thickness between about 0.01 inches to about 0.013 inches.

### Adhesion Test Procedure

In this adhesion force test, approximately 0.50 g of a liner sample (cut into a square inch piece) is placed on the center of the lower platform (which is surrounded on the edges by a specially cut petri dish) of the Chatillon gauge Model TCD200. The Chatillon instrument simulates mastication while the liner sample is completely submerged in water. Instead of an application of continual force, the distances are constant and repetitive. Lower the Chatillon ram until a slight force is felt between the head and the platform. Adjust the ram so that only a force between 0.00 and 0.10 lb. is achieved. This is the zero point. The height of the head is set at 0.20 mm above the base. Pour 50 gm of deionized water into the petri dish. The machine executes 250 simulations during one test run, moving up and down a distance of 0.10 mm from the set point. The duration of one test run is 45 minutes. Adhesion/compression forces in lbs. are recorded and plotted graphically.

### Dislodge Force Procedure

For the dislodge force test, 0.50 gm of a liner sample is placed on a removable lower acrylic plate. The custom made apparatus measures the force required to separate the tested material from the acrylic plate to which the test material is adhered. A removable upper acrylic plate is covered with a piece of cloth dampened with 1 ml. of deionized water. The chamber is filled with deionized water (enough to completely submerge the upper and lower plates) kept at a constant temperature of 37° C. within the internal chamber of the water bath. There is an air compression force of 10 lbs. applied for 5 minutes. After 5 minutes, air compression is released and the lower plate pulls away. The amount of force required to separate the plates is measured in lbs. The test is repeated at 5 minute intervals (a reading is taken every 5 minutes) for a period of 60 minutes. Dislodge force in lbs. is recorded and plotted graphically. The higher the force required to separate the upper and lower plates, the better the denture adhesive performance.

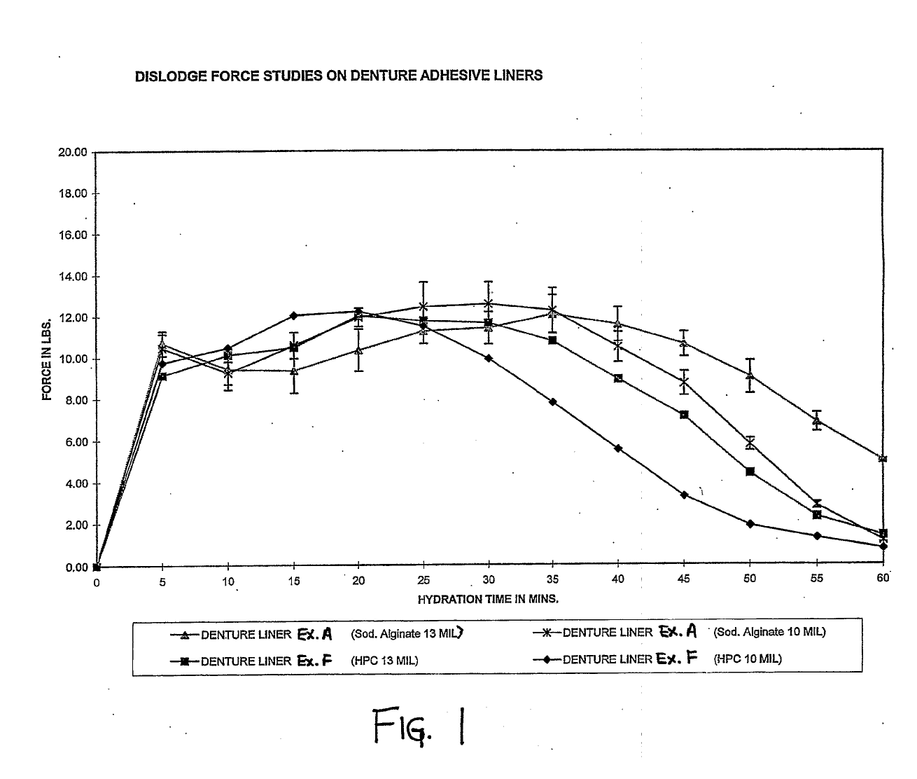

Referring to FIG. 1, it is clear that a denture liner according to the present invention (Ex. A) exhibits comparable initial dislodge force properties to the comparative liner (Ex. F) and superior dislodge force properties after a 30 minute hydration time.

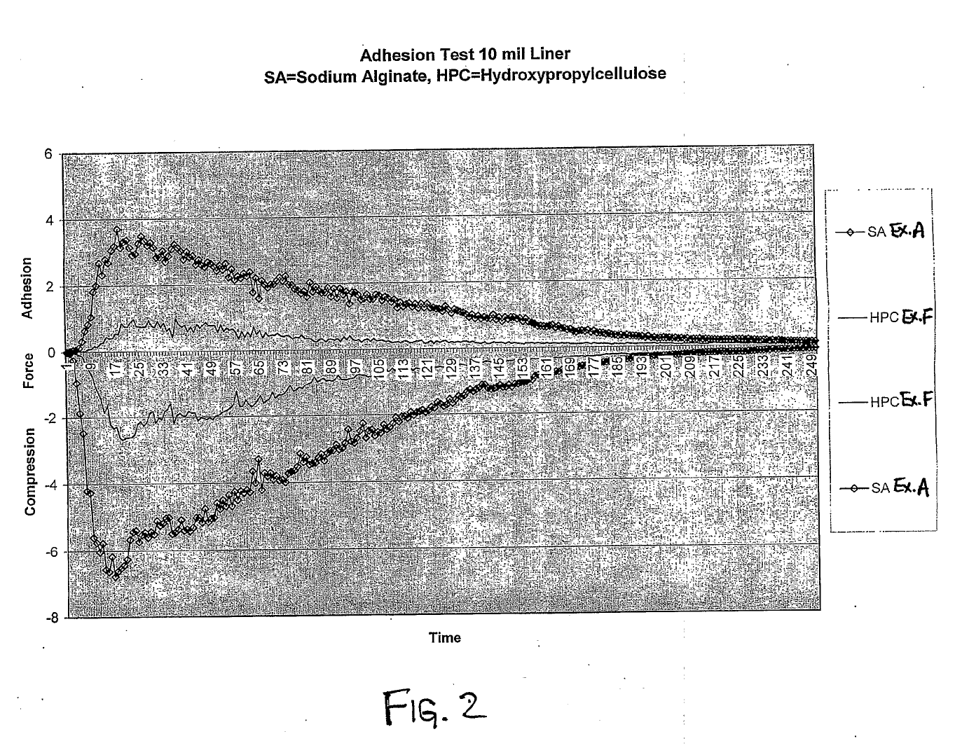

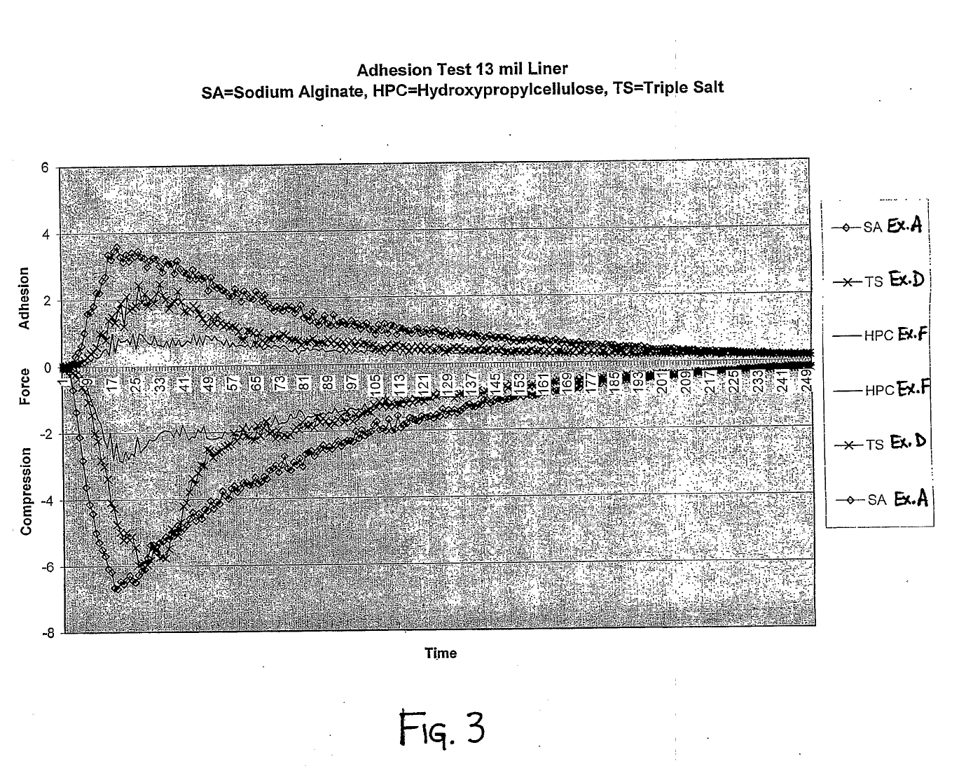

Referring to FIGS. 2 and 3, it is clearly demonstrated that adhesive liners according to the present invention (Ex. A) possess superior adhesion and compression force characteristics than those of the comparative liners (Ex. D and Ex. F).

The present invention also provides a method for adhering a denture to a user's mouth. The method includes the steps of (a) applying one or more denture adhesive liners according to the present invention to the denture; and (b) positioning the denture in the user's mouth. Upon contact with the wet mucosa surface in the user's mouth, the denture adhesive liner becomes adhesive and adheres the denture to the user's mouth.

The above describes the invention including preferred embodiments thereof. It should be understood that modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A denture adhesive liner composition consisting essentially of: (a) an adhesive component selected from the group consisting of one or more alginates present in a denture adhesive effective amount; (b) thermoplastic polymer component; and (c) plasticizer; wherein said composition is extrudable into a denture adhesive liner in the form of a film or sheet that is capable of adhering to a wet mucous surface.

2. The denture adhesive liner composition of claim 1, wherein said one or more alginates is selected from the group consisting of algin, alginic acid, alginate salt, sodium alginate, potassium alginate, calcium alginate, magnesium alginate, propylene glycol alginate, triethanolamine alginate, and any combinations thereof.

3. The denture adhesive liner composition of claim 1, wherein said denture adhesive effective amount is about 5 wt. % to about 60 wt. %, based on the total weight of said composition

4. The denture adhesive liner composition of claim 1, wherein said thermoplastic polymer component is selected from the group consisting of polyethylene oxide, hydroxypropylcellulose, hydroxypropylmethylcellulose, and any combinations thereof.

5. The denture adhesive liner composition of claim 1, wherein said thermoplastic polymer component is present in an amount of from about 30 wt. % to about 90 wt. %, based on the total weight of the denture adhesive liner composition.

6. The denture adhesive liner composition of claim 1, wherein said thermoplastic polymer component is present in an amount of from about 70 to about 80 wt. %, based on the total weight of the denture adhesive liner composition.

7. The denture adhesive liner composition of claim 1, wherein said plasticizer is selected from the group consisting of water, polyethylene oxide, polypropylene oxide, glycol, propylene glycol, polyethylene glycol, polyhydric alcohol, glycerin, sorbitol, glycerol ester, glycerol triacetate, fatty acid triglyceride, naphthenic oil, aromatic oil, vegetable oil, castor oil, low molecular weight rosin ester, polyterpene, or any combinations thereof.

8. The denture adhesive liner composition of claim 1, wherein said plasticizer is present in an amount from about 1 wt. % to about 30 wt. %, based on the total weight of the denture adhesive liner composition.

9. The denture adhesive liner composition of claim 1, wherein said plasticizer is present in an amount from about 3 wt. % to about 6 wt. %, based on the total weight of the denture adhesive liner composition.

10. The denture adhesive liner composition of claim 1, wherein said denture adhesive liner is a single-layer extruded film or foam sheet.

11. The denture adhesive liner composition of claim 1, further comprising from about 0 to about 30 wt. %, based on the total weight of the denture adhesive composition, of one or more therapeutic actives.

12. The denture adhesive liner composition of claim 11, wherein said one or more therapeutic actives is selected from the group consisting of iodine, sulfonamides, bisbiguanides, phenolics, antibiotics, tetracycline, neomycin, kanamycin, metronidazole, clindamycin, anti-inflammatory agents, aspirin, acetaminophen, naproxen, naproxen salts, ibuprofen, ketorolac, flurbiprofen, indomethacin, cimetidine, eugenol, hydrocortisone, dentinal desensitizing agents, potassium nitrate, potassium chloride, strontium chloride, sodium fluoride, anesthetic agents, lidocaine, benzocaine, anti-fungals, aromatics, camphor, eucalyptus oil, aldehyde derivatives, benzaldehyde, insulin, steroids, anti-neoplastics, and any combinations thereof.

13. The denture adhesive liner composition of claim 1, further comprising from about 0 wt. % to about 30 wt. %, based on the total weight of the composition, of one or more odor adsorbent components.

14. The denture adhesive liner composition of claim 13, wherein said one or more odor adsorbing components is selected from the group consisting of α-cyclodextrin, β-cyclodextrin and γ-cyclodextrin carboxylmethyl cyclodextrin, hydroxypropyl cyclodextrin, methyl cyclodextrin, sulfated cyclodextrin, quaternary ammonium cyclodextrin, activated carbon, sodium carbonate, and any combinations thereof.

15. The denture adhesive liner composition of claim 1, further comprising one or more additional components selected from the group consisting of silicon dioxide, antioxidant, ascorbic acid, Vitamin E, colorant, preservative, methyl paraben, propyl paraben, thickener, flavor, fragrance, sensate enhancer, and any combinations thereof.

16. A method for preparing a denture adhesive liner comprising the steps of: (a) preparing a composition consisting essentially of: (i) a denture adhesive effective amount of a denture adhesive selected from the group consisting of one or more alginates; (ii) a thermoplastic polymer component; and (iii) a plasticizer; and (b) forming a denture adhesive liner from said composition by extruding said composition through an extruder under increased pressure through a die such that it forms a film or sheet.

17. The method of claim 16, wherein said denture adhesive liner is in the form of a single-layer extruded film or foam sheet.

18. The method of claim 16, wherein said denture adhesive liner is extruded using a co-extruder forming a multi-layer extruded film.

19. The method of claim 16, wherein said thermoplastic polymer component is selected from the group consisting of polyethylene oxide, hydroxypropylcellulose, hydroxypropylmethylcellulose, and any combinations thereof.

20. The method of claim 16, wherein said thermoplastic polymer component is present in an amount of from about 50 to about 90 wt. %, based on the total weight of the composition.

21. The method of claim 16, wherein said plasticizer is selected from the group consisting of water, polyethylene oxide, polypropylene oxide, glycol, propylene glycol, polyethylene glycol, polyhydric alcohol, glycerin, sorbitol, glycerol ester, glycerol triacetate, fatty acid triglyceride, naphthenic oil, aromatic oil, vegetable oil, castor oil, low molecular weight rosin ester, polyterpene, or any combinations thereof.

22. The method of claim 16, wherein said plasticizer is present in an amount from about 1 wt. % to about 30 wt. %, based on the total weight of the composition.

23. The method of claim 16, wherein said composition further comprises from about 0 to about 30 wt.-%, based on the total weight of the composition, of one or more therapeutic actives selected from the group consisting of iodine, sulfonamides, bisbiguanides, phenolics, antibiotics, tetracycline, neomycin, kanamycin, metronidazole, clindamycin, anti-inflammatory agents, aspirin, acetaminophen, naproxen, naproxen salts, ibuprofen, ketorolac, flurbiprofen, indomethacin, cimetidine, eugenol, hydrocortisone, dentinal desensitizing agents, potassium nitrate, potassium chloride, strontium chloride, sodium fluoride, anesthetic agents, lidocaine, benzocaine, anti-fungals, aromatics, camphor, eucalyptus oil, aldehyde derivatives, benzaldehyde, insulin, steroids, anti-neoplastics, and any combinations thereof.

24. The method of claim 16, wherein said composition further comprises from about 0 wt. % to about 20 wt. %, based on the total weight of the composition, one or more odor adsorbent components selected from the group consisting of α-cyclodextrin, β-cyclodextrin and γ-cyclodextrin carboxylmethyl cyclodextrin, hydroxypropyl cyclodextrin, methyl cyclodextrin, sulfated cyclodextrin, quaternary ammonium cyclodextrin, activated carbon, sodium bicarbonate, and any combinations thereof.

25. The method of claim 16, wherein said composition further comprises one or more additional components selected from the group consisting of silicon dioxide, antioxidant, ascorbic acid, Vitamin E, colorant, preservative, methyl paraben, propyl paraben, thickener, flavor, fragrance, sensate enhancer, and any combinations thereof.

26. A method for adhering a denture to a user's mouth comprising the steps of (a) applying one or more denture adhesive liners consisting essentially of an adhesive component selected from the group consisting of one or more alginates present in a denture adhesive effective amount; thermoplastic polymer component; and plasticizer, to said denture; and (b) positioning said denture in said user's mouth,
wherein said one or more denture adhesive liners become adhesive upon contact with a wet mucosa surface in said user's mouth thereby adhering said denture to said user's mouth.
